(19) United States
(12) Patent Application Publication
(10) Pub. No.: US 2010/0047130 A1
(43) Pub. Date: Feb. 25, 2010

(54) Lab-On-Cd Systems With Magnetically Actuated Micro Check Valves And/Or Magnetic Immobilization

(75) Inventors: Eng Hoo Teddy Ong, Singapore (SG); Vinayak Ashok Prabhu, Singapore (SG); Teck Boon Yap, Singapore (SG)

Correspondence Address: LAWRENCE Y.D. HO & ASSOCIATES PTE LTD, 30 BIDEFORD ROAD, #02-02, THONGSIA BUILDING, SINGAPORE, 229922, SG

(73) Assignee: Nanyang Polytechnic, Singapore (SG)

(21) Appl. No.: 12/446,753

(22) PCT Filed: Oct. 19, 2007
(86) PCT No.: PCT/SG07/00359

§ 371 Date: Apr. 23, 2009

(30) Foreign Application Priority Data
Oct. 25, 2006 (SG) 200607398-5

(51) Int. Cl. G01N 33/50

(52) U.S. Cl. 422/68.1

(57) ABSTRACT

The present invention provides lab-on-CD (LoCD) systems for conducting chemical and biological reactions. One of the LoCD systems comprises a microfluidic CD with at least one magnetically actuated micro check valve, said microfluidic CD having at least one sample reservoir, at least one reaction chamber, and at least one microfluidic channel connecting the at least one sample reservoir and the at least one reaction chamber; wherein the at least one magnetically actuated micro check valve is positioned to control the microfluidic flow in the at least one microfluidic channel; and a supporting CD with at least one magnetic element for providing a magnetic force; thereby when the microfluidic CD and the supporting CD are assembled into the LoCD system, the at least one magnetic element can move the at least magnetically actuated micro check valve so as to control the microfluidic flow in the at least one microfluidic channel.

### FIELD OF THE INVENTION

The present invention generally relates to technologies for miniaturization devices for carrying out biological or chemical analyses, and more particularly to lab-on-Compact Disc (CD) systems with magnetically actuated micro check valves and/or magnetic immobilization.

### BACKGROUND OF THE INVENTION

The advances of miniaturizing systems/devices for carrying out biological or chemical analyses have led to lab-on-chip and lab-on-CD platforms. In both platforms, sample reservoirs and reaction chambers are connected by a network of microfluidic channels. However, the control and transport of reaction samples and buffers have been challenges for the design and fabrication of the lab-on-chip and lab-on-CD systems. For conventional lab-on-chip systems, micropumps have been used to control and transport the fluidic transport. In contrast, the lab-on-CD systems use the inherent centrifugal forces of the rotating CD to drive the fluids through microfluidic channels on its surface for various biological reactions. In addition, the CD format enables the lab-on-CD systems to be adaptable in various CD devices including computer and CD players. For example, U.S. Pat. No. 6,030,581 discloses a lab-on-CD system that enables a user to carry out biological analyses on a computer.

The flow control on a rotating lab-on-CD platform is designed by employing passive valves on selective radial locations on the CD. Depending on this radial location and the geometrical shape and size of the passive valve, the rotation frequency (RPM) at which the valve allows flow (burst frequency) will be determined. The performance of passive valves is heavily dependant on both the design and process parameters. The design parameters include valve dimensions and radial position and the process parameters include surface characteristics and process variations on valve dimensions. These factors make the valve performance unpredictable and not effectively reproducible. The valve leakage and back flow also play a part in decreasing its efficiency.

In order to effectively use of this centrifugal force mechanism, efficient valves are needed to control fluid flow and program the on/off positions according to the application needs. These valves need to operate only under designed rotation frequencies in RPM with minimum leakage. Also under special conditions, at particular application nodes on the lab-on-CD, one might require several valves to operate in a programmed manner from a common location. These flow controls pose a problem when not operating efficiently to lab-on-CD microfluidics.

There are many designs and configurations of the valves used in a lab-on-CD system. For example, U.S. Pat. No. 6,030,581 discloses a valve that is made from a thin gold coil for controlling two capillaries via two electrodes. In addition, it discloses that valve-like operations may be performed chemically by deposition from solution of a solid chemical compound and/or dissolution of a deposited, solid compound. However, all are complicated and complex.

Another problem facing the lab-on-CD systems is the immobilization of bio-molecules to the reaction chamber. For example, there is a lack of immobilization methods to probe nucleic acid molecules like DNA on specific reaction sites on the lab-on-CD. The immobilization method used must be able to withstand the high rotation speed and the centrifugal force caused by it in order to avoid being washed away by the flowing fluid.

### SUMMARY OF THE INVENTION

One embodiment of the present invention provides a lab-on-CD (LoCD) system for conducting chemical and biological reactions. The LoCD system comprises a microfluidic CD with at least one magnetically actuated micro check valve, said microfluidic CD having at least one sample reservoir, at least one reaction chamber, and at least one microfluidic channel connecting the at least one sample reservoir and the at least one reaction chamber; wherein the at least one magnetically actuated micro check valve is positioned to control the microfluidic flow in the at least one microfluidic channel; and a supporting CD with at least one magnetic element for providing a magnetic force; thereby when the microfluidic CD and the supporting CD are assembled into the LoCD system, the at least one magnetic element can move the at least magnetically actuated micro check valve so as to control the microfluidic flow in the at least one microfluidic channel.

In another embodiment of the LoCD system, the at least one magnetically actuated micro check valve is a metallic micro object. In a further embodiment of the LoCD system, the metallic micro object has a spherical configuration with a diameter less than 1 mm.

In another embodiment of the LoCD system, the at least one magnetic element is a permanent magnet, an electromagnet, or any other suitable magnetic means.

In another embodiment of the LoCD system, the microfluidic CD is more than one so that they can be stacked together and controlled simultaneously by the supporting CD.

In another embodiment of the LoCD system, the supporting CD further comprises a plurality of central latch arms for providing convenience for assembling the LoCD system.

In another embodiment of the LoCD system, it further comprises a central shaft attach support that is configured to be complementary with the center part of the supporting CD.

In another embodiment of the LoCD system, the microfluidic CD further comprises at least one magnetic element embedded under the at least one reaction chamber; thereby the at least one magnetic element can immobilize magnetic beads to the bottom surface of the at least one reaction chamber, thus when the magnetic beads are coated with a molecule specific for one entity in a sample mix, the one entity can be isolated from the sample mix with the immobilized magnetic beads.

In another embodiment of the LoCD system, the supporting CD further comprises at least another magnetic element; thereby the at least another magnetic element can be reversibly positioned under the reaction chamber so that magnetic beads from a sample mix can be immobilized or released from the bottom surface of the reaction chamber.

In another embodiment of the LoCD system, the at least one magnetic element can be reversibly positioned under the reaction chamber so that magnetic beads from a sample mix can be immobilized or released from the bottom surface of the reaction chamber.

Another embodiment of the present invention provides a lab-on-CD (LoCD) system for conducting chemical and biological reactions. The LoCD system comprises a microfluidic CD having at least one sample reservoir, at least one reaction chamber, at least one microfluidic channel connecting the at least one sample reservoir and the at least one reaction chamber, and at least one magnetic element embedded under the at least one reaction chamber; thereby magnetic beads from a sample mix can be immobilized or released from the bottom surface of the reaction chamber. In another embodiment of the lab-on-CD (LoCD) system, the at least magnetic element is permanent magnet, an electromagnet, or any other suitable magnetic means. In yet another embodiment of the lab-on-CD (LoCD) system, the bottom surface of the reaction chamber can be roughed.

Another embodiment of the present invention provides a lab-on-CD (LoCD) system for conducting chemical and biological reactions. The LoCD system comprises a microfluidic CD having at least one sample reservoir, at least one reaction chamber, at least one microfluidic channel connecting the at least one sample reservoir and the at least one reaction chamber, and at least one magnetic element embedded under the at least one reaction chamber; and a supporting CD with at least one magnetic element for providing a magnetic force; thereby when the microfluidic CD and the supporting CD are assembled into the LoCD system, the at least one magnetic element can be reversibly positioned under the reaction chamber so that magnetic beads from a sample mix can be immobilized or released from the bottom surface of the reaction chamber. In another embodiment of the lab-on-CD (LoCD) system, the at least magnetic element is permanent magnet, an electromagnet, or any other suitable magnetic means. In yet another embodiment of the lab-on-CD (LoCD) system, the bottom surface of the reaction chamber can be roughed.

The present invention has many advantages over the existing LoCD systems. For example, metallic micro objects as a check valve can be used inline the microchannel, at the outlet of a micro reservoir or at the inlet of a reaction chamber. In addition, the metallic micro objects can be actuated by magnetic forces that can be generated by permanent magnets or electro magnets. Furthermore, multiple microfluidic CDs can be simultaneously controlled by one magnetic force. Finally, the magnetic element can be used to immobilize magnetic beads.

### BRIEF DESCRIPTION OF THE DRAWINGS

Preferred embodiments according to the present invention will now be described with reference to the Figures, in which like reference numerals denote like elements.

FIG. 1 is a schematic diagram of a lab-on-CD (LoCD) system in accordance with one embodiment of the present invention.

FIG. 2 is a superimposed view of the LoCD system shown in FIG. 1.

FIGS. 3(a)-(d) show four exemplary configurations of in-reservoir electro/magnetic flow check valve.

FIGS. 4(a)-(b) show schematic diagrams showing the operation of electro/magnetic flow check valve in accordance with one embodiment of the present invention.

FIGS. 5(a)-(c) show cross-sectional diagrams showing the operation of electro/magnetic flow check valve in accordance with one embodiment of the present invention.

FIG. 6 is an exploded view of a LoCD system with immobilization in accordance with one embodiment of the present invention.

FIGS. 7(a)-(e) are schematic diagrams showing the reversible immobilization of bio-molecules in a LoCD system in accordance with one embodiment of the present invention.

FIGS. 8(a)-(d) are schematic diagrams showing the reaction chamber with embedded magnetic element and its immobilization of biomolecules in accordance with one embodiment of the present invention.

FIG. 9 is a superimposed view of a magnetic disc/strip aligned with the reaction chamber in accordance with one embodiment of the present invention.

FIG. 10 shows the bottom surface of the reaction chamber that can be roughed in order to facilitate the formation of magnetic bead matrix.

### DETAILED DESCRIPTION OF THE INVENTION

The present invention may be understood more readily by reference to the following detailed description of certain embodiments of the invention.

Throughout this application, where publications are referenced, the disclosures of these publications are hereby incorporated by reference, in their entirety, into this application in order to more fully describe the state of art to which this invention pertains.

In the following detailed description, specific details are set forth in order to provide a thorough understanding of the invention. However, it will be understood by those skilled in the relevant art that the present invention may be practiced without these specific details. In other instances, well-known methods, procedures, components, and materials have not been described in detail so as not to obscure the present invention.

The present invention provides lab-on-CD systems that have improved features over existing systems. Briefly, one feature is that the valves for controlling the fluidic flows have simple designs and can be easily controlled. Another feature is that the bio-molecules in reaction samples can be easily and selectively immobilized or released onto or from the bottom surface of the reaction chamber.

One embodiment of present invention provides a lab-on-CD system that comprises a microfluidic CD with at least one magnetically actuated micro check valve, and a supporting CD. The magnetically actuated micro check valve may be a metallic micro object that may be spherical or otherwise with a diameter less than 1 mm, blocking the microfluidic channels in the microfluidic CD whenever flow is not required and being displaced away when flow is needed. The micro object is actuated by magnetic force. The magnetic force is provided by permanent magnets or electromagnets or any other suitable magnetic means that are embedded in the actuating CD that rotates relative to the microfluidic CD to trigger the micro object movement. In certain embodiments, the actuating CD is able to simultaneously control multiple stacked microfluidic CDs.

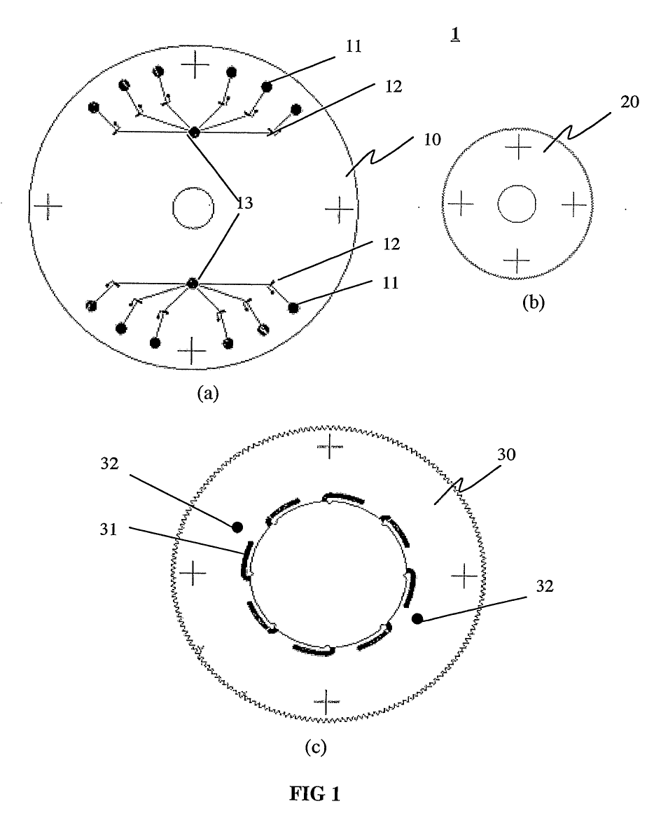

Now referring to FIG. 1, there is provided a schematic diagram of a lab-on-CD (LoCD) system in accordance with one embodiment of the present invention. The LoCD system 1 comprises a microfluidic CD 10 shown in FIG. 1(a), and a supporting CD 30 shown in FIG. 1(c). The microfluidic CD 10 with a circular configuration comprises at least one sample inlet/reservoir chamber 11, at least one reaction chamber 13, and at least one channel via a micro check valve 12 connecting the at least one sample inlet/reservoir chamber 11 and the at least one reaction chamber 13. The design and fabrication of the microfluidic CD is well known in the art; thus no more details will be provided herein. The supporting CD 30 comprises a plurality of central latch arms 31 and at least one embedded actuating magnet 32. The central latch arms 31 provide convenience for assembling the LoCD system. The embedded actuating magnet 32 controls the positions of the micro check valve 12 in the microfluidic CD 10.

Still referring to FIG. 1, the LoCD system 1 further comprises a central shaft attach support 20 shown in FIG. 1(b). The central shaft attach support 20 is configured to be complementary with the center part of the supporting CD 30. The latching arms of the supporting CD latch onto the central shaft attach providing a spring-like mechanism. This spring-like mechanism allows the supporting CD to rotate even when the central shaft is stationary. However, when the central shaft rotates, the latching arms facilitate the rotation of the supporting CD in sync with the central shaft.

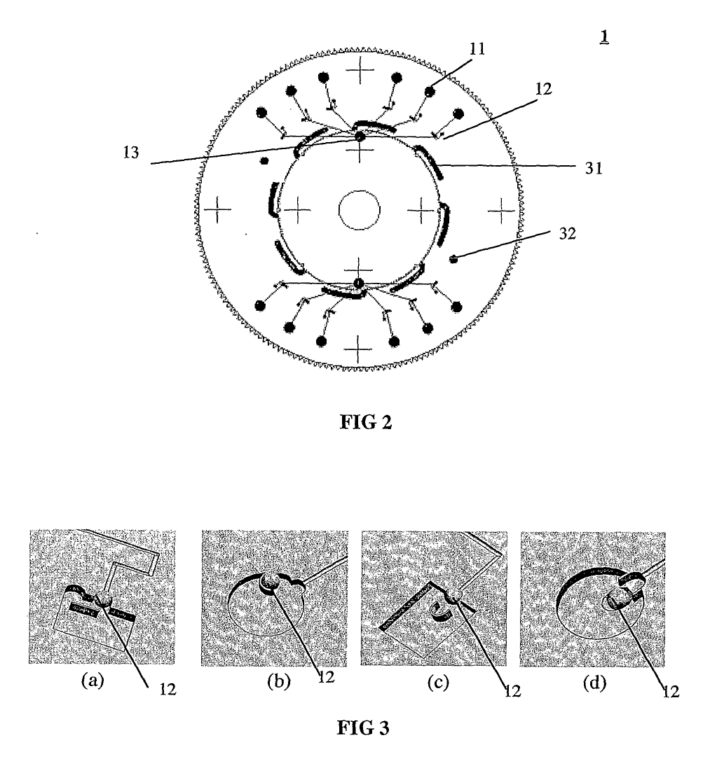

Referring to FIG. 2, there is provided a superimposed view of the LoCD system shown in FIG. 1. It is to be noted that the embedded actuating magnet 32 is not overlapped with the micro check valve 12 in FIG. 1. The operations of controlling the micro check valve will be described hereinafter.

Referring to FIGS. 3(a)-(d), there are provided four exemplary configurations of in-reservoir electro/magnetic flow micro check valve 12.

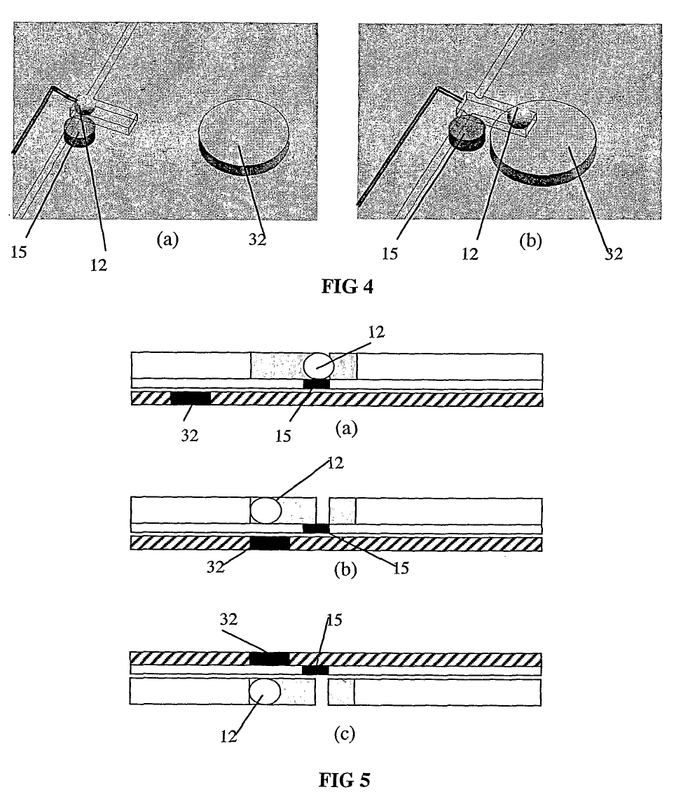

Referring to FIGS. 4(a)-(b), there are provided schematic diagrams showing the operation of the electro/magnetic flow micro check valve in accordance with one embodiment of the present invention. The electro/magnetic flow check valve 12 is in OFF state when the micro check valve 12 is positioned in line of the communicating channel by the small embedded magnet 15 in the microfluidic CD as shown in FIG. 4(a). The electro/magnetic flow micro check valve 12 is in ON state when the micro check valve 12 is positioned out line of the communicating channel by the bigger actuating magnet 32 embedded in the supporting CD when the actuating magnet 32 is moved closer to the micro check valve 12 as shown in FIG. 4(b). It is to be noted that the actual controlling the position of the micro check valve by the actuating magnet is not limited to the one shown in FIG. 4.

Referring to FIGS. 5(a)-(c), there are provided cross-sectional diagrams showing the operation of electro/magnetic flow check valve in accordance with one embodiment of the present invention. The electro/magnetic flow micro check valve 12 is in OFF state when the micro check valve 12 is positioned in line of the communicating channel by the small embedded magnet 15 in the microfluidic CD as shown in FIG. 5(a). The electro/magnetic flow micro check valve 12 is in ON state when the micro check valve 12 is positioned out line of the communicating channel by the bigger actuating magnet 32 embedded in the supporting CD when the actuating magnet 32 moves closer to the micro check valve 12 as shown in FIG. 5(b). FIG. 5(c) shows another configuration of the micro check valve and actuating magnet where the actuating magnet 32 is above the micro check valve 12.

Another embodiment of the present invention provides a lab-on-CD system that utilizes magnetic forces to immobilize reaction reagents including biomolecules such as DNA and proteins. The magnetic forces may be provided by a permanent or movable magnetic element that is aligned with reaction chambers. The aligned magnetic element enables to immobilize magnetic beads. For example, the streptavidin coated micro beads are attracted by an embedded magnetic disc that covers the entire area of the reaction chamber. The streptavidin beads then hold the bio-molecules thereby conjugating them inside the reaction chamber. The magnetic force is large enough to hold these beads with the nucleic acids bound to them even when the CD is rotating at high RPM speeds.

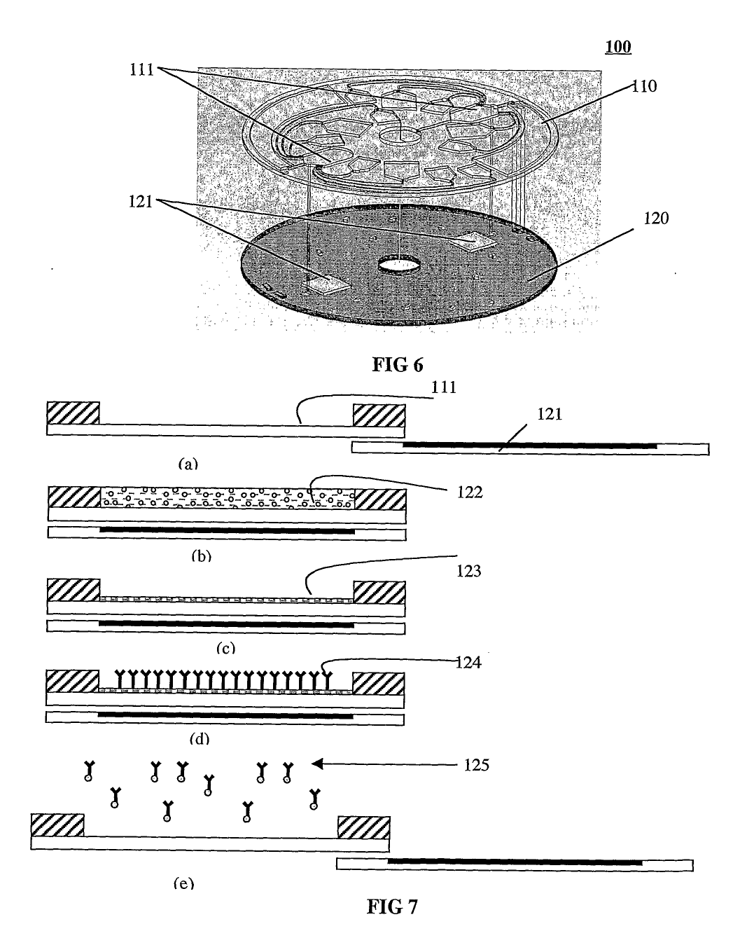

Referring to FIG. 6, there is provided an exploded view of a LoCD system with embedded magnetic element in the supporting CD in accordance with one embodiment of the present invention. As shown in FIG. 6, the LoCD system 100 comprises a microfluidic CD 110 having a plurality of reaction chambers 111, and a supporting CD 120 having a plurality of magnetic elements 121 embedded therein. As will be described more in detail hereinafter, the embedded magnetic elements 121 in the supporting CD can be aligned with the reaction chambers to provide magnetic forces so as to immobilize magnetic beads.

Referring to FIGS. 7(a)-(e), there are provided schematic diagrams showing the reversible immobilization of bio-molecules in a LoCD system in accordance with one embodiment of the present invention. As shown in FIG. 7(a), the reaction chamber 111 does not have any embedded magnetic material; instead a magnetic element 121 is embedded in the supporting CD that can be reversibly positioned underneath of the reaction chamber. After the LoCD system is assembled, the magnetic element 121 embedded in the supporting CD is aligned underneath with the reaction chamber 111; then the reaction chamber 111 is filled with buffer mix 122 containing bio-coated micro/nano magnetic beads shown in FIG. 7(b); and then the micro/nano magnetic beads are attracted by the magnetic element 121 to form a layered matrix 123 on the bottom of the reaction chamber shown in FIG. 7(c). Then, the molecules attached to the micro/nano magnetic beads will be immobilized onto the bottom of the reaction chamber. Alternatively, bio-molecules that are capable of binding to the bio-coating of the micro/nano magnetic beads 124 can be immobilized indirectly via binding to the bio-coated micro/nano magnetic beads as shown in FIG. 7(d). Finally, the magnetic disc/strip embedded in the supporting CD can be moved away from the reaction chamber, so that the micro/nano magnetic beads 125 are released from the bottom of the reaction chamber shown in FIG. 7(e).

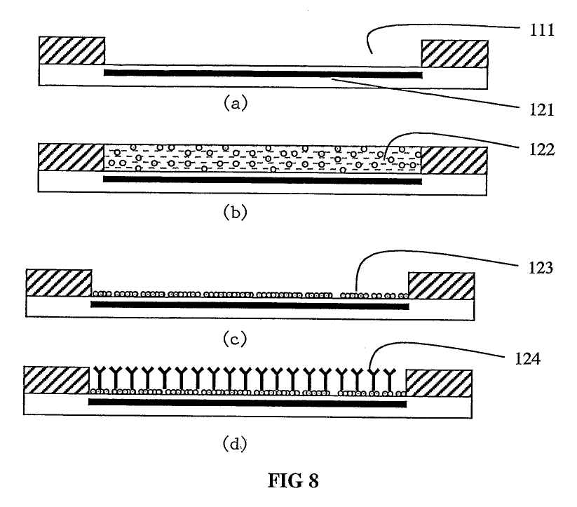

Referring to FIGS. 8(a)-(d), there is provided a schematic diagram showing the reaction chamber with an embedded magnetic element and its immobilization of biomolecules in accordance with one embodiment of the present invention. As shown in FIG. 8(a), the reaction chamber 111 comprises an embedded magnetic element 121 underneath of the reaction chamber. The embedded magnetic element 121 can be made of permanent magnetic materials. When the reaction chamber 111 is filled with a buffer mix 122 containing bio-coated micro/nano magnetic beads shown in FIG. 8(b), the micro/nano magnetic beads are attracted by the magnetic disc/strip to form a layered matrix 123 on the bottom of the reaction chamber shown in FIG. 7(c). Then, the molecules 124 attached to the micro/nano magnetic beads will be immobilized onto the bottom of the reaction chamber. Alternatively, bio-molecules that are capable of binding to the bio-coating of the micro/nano magnetic beads can be immobilized indirectly via binding to the bio-coated micro/nano magnetic beads as shown in FIG. 7(d).

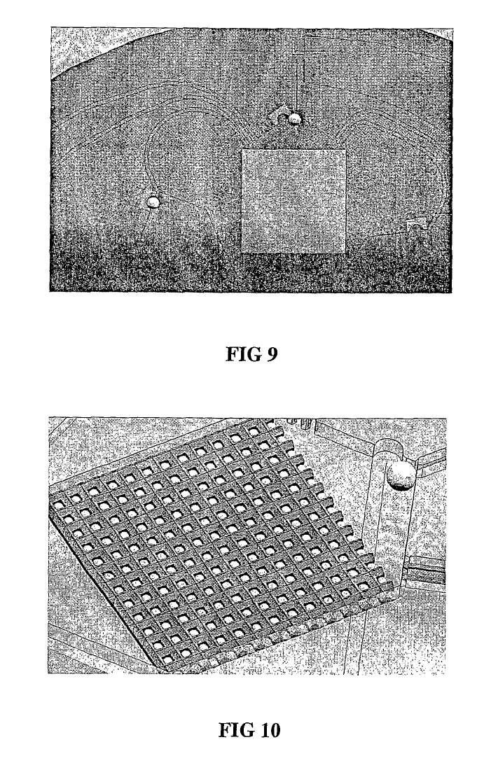

Referring to FIG. 9, there is provided a superimposed view of magnetic elements aligned with the reaction chamber in accordance with one embodiment of the present invention.

In order to facilitate of forming a matrix on the bottom of the reaction chamber by the micro/nano magnetic beads, the bottom surface of the reaction chamber can be roughed as shown in FIG. 10.

Another embodiment of the present invention provides a LoCD system that utilizes the micro check valves to control the microfluidic flow and employs the magnetic forces to immobilize the reaction reagents as discussed above. These two features can be combined in any suitable manner; thus no details of such a combination will be provided herein.

While the present invention has been described with reference to particular embodiments, it will be understood that the embodiments are illustrative and that the invention scope is not so limited. Alternative embodiments of the present invention will become apparent to those having ordinary skill in the art to which the present invention pertains. Such alternate embodiments are considered to be encompassed within the spirit and scope of the present invention. Accordingly, the scope of the present invention is described by the appended claims and is supported by the foregoing description.

What is claimed is:

1. A lab-on-CD (LoCD) system for conducting chemical and biological reactions, comprising:
a microfluidic CD with at least one magnetically actuated micro check valve, said microfluidic CD having at least one sample reservoir, at least one reaction chamber, and at least one microfluidic channel connecting the at least one sample reservoir and the at least one reaction chamber; wherein the at least one magnetically actuated micro check valve is positioned to control the microfluidic flow in the at least one microfluidic channel; and
a supporting CD with at least one magnetic element for providing a magnetic force;
thereby when the microfluidic CD and the supporting CD are assembled into the LoCD system, the at least one magnetic element can move the at least magnetically actuated micro check valve so as to control the microfluidic flow in the at least one microfluidic channel;
wherein the microfluidic CD further comprises at least one magnetic element embedded under the at least one reaction chamber; thereby the at least one magnetic element can immobilize magnetic beads to the bottom surface of the at least one reaction chamber, thus when the magnetic beads are coated with a molecule specific for one entity in a sample mix, the one entity can be isolated from the sample mix with the immobilized magnetic beads.

2. The LoCD system of claim 1, wherein the at least one magnetically actuated micro check valve is a metallic micro object.

3. The LoCD system of claim 2, wherein the metallic micro object has a spherical configuration with a diameter less than 1 mm.

4. The LoCD system of claim 1, wherein the at least one magnetic element is a permanent magnet, an electromagnet, or any other suitable magnetic means.

5. The LoCD system of claim 1, wherein the microfluidic CD is more than one so that they can be stacked together and controlled simultaneously by the supporting CD.

6. The LoCD system of claim 1, wherein the supporting CD further comprises a plurality of central latch arms for providing convenience for assembling the LoCD system.

7. The LoCD system of claim 1, further comprising a central shaft attach support that is configured to be complementary with the center part of the supporting CD.

8. A lab-on-CD (LoCD) system for conducting chemical and biological reactions, comprising:
a microfluidic CD with at least one magnetically actuated micro check valve, said microfluidic CD having at least one sample reservoir, at least one reaction chamber, and at least one microfluidic channel connecting the at least one sample reservoir and the at least one reaction chamber; wherein the at least one magnetically actuated micro check valve is positioned to control the microfluidic flow in the at least one microfluidic channel; and
a supporting CD with at least one magnetic element for providing a magnetic force;
thereby when the microfluidic CD and the supporting CD are assembled into the LoCD system, the at least one magnetic element can move the at least magnetically actuated micro check valve so as to control the microfluidic flow in the at least one microfluidic channel;
wherein the supporting CD further comprises at least another magnetic element; thereby the at least another magnetic element can be reversibly positioned under the reaction chamber so that magnetic beads from a sample mix can be immobilized or released from the bottom surface of the reaction chamber.

9. A lab-on-CD (LoCD) system for conducting chemical and biological reactions, comprising:
a microfluidic CD with at least one magnetically actuated micro check valve, said microfluidic CD having at least one sample reservoir, at least one reaction chamber, and at least one microfluidic channel connecting the at least one sample reservoir and the at least one reaction chamber; wherein the at least one magnetically actuated micro check valve is positioned to control the microfluidic flow in the at least one microfluidic channel; and
a supporting CD with at least one magnetic element for providing a magnetic force;
thereby when the microfluidic CD and the supporting CD are assembled into the LoCD system, the at least one magnetic element can move the at least magnetically actuated micro check valve so as to control the microfluidic flow in the at least one microfluidic channel;
wherein the at least one magnetic element can be reversibly positioned under the reaction chamber so that magnetic beads from a sample mix can be immobilized or released from the bottom surface of the reaction chamber.

10. A lab-on-CD (LoCD) system for conducting chemical and biological reactions, comprising:
a microfluidic CD having at least one sample reservoir, at least one reaction chamber, at least one microfluidic channel controllably connecting the at least one sample reservoir and the at least one reaction chamber, and at least one magnetic element embedded underneath of the at least one reaction chamber; thereby magnetic beads from a sample mix can be immobilized onto or released from the bottom surface of the reaction chamber.

11. The lab-on-CD (LoCD) system of claim 11, wherein the embedded at least one magnetic element is permanent magnet, an electromagnet, or any other suitable magnetic means.

12. The lab-on-CD (LoCD) system of claim 11, wherein the bottom surface of the reaction chamber can be roughed.

13. A lab-on-CD (LoCD) system for conducting chemical and biological reactions, comprising:
a microfluidic CD having at least one sample reservoir, at least one reaction chamber, at least one microfluidic channel controllably connecting the at least one sample reservoir and the at least one reaction chamber; and
a supporting CD having at least one embedded magnetic element;
thereby when the microfluidic CD and the supporting CD are assembled into the LoCD system the at least one embedded magnetic element in the supporting CD can be reversibly positioned under the reaction chamber so that magnetic beads from a sample mix can be immobilized onto or released from the bottom surface of the reaction chamber.

14. The lab-on-CD (LoCD) system of claim 14, wherein the at least one embedded magnetic element is permanent magnet, an electromagnet, or any other suitable magnetic means.

15. The lab-on-CD (LoCD) system of claim 14, wherein the bottom surface of the reaction chamber can be roughed.
